(19) United States
(12) Patent Application Publication
(10) Pub. No.: US 2026/0077103 A1
(43) Pub. Date: Mar. 19, 2026

(54) DEMINERALIZED BONE FIBER IMPLANT COMPOSITION FOR CARTILAGE REPAIR

(71) Applicant: TETROUS, INC., Sherman Oaks, CA (US)

(72) Inventors: Andrew J. CARTER, June Lake, CA (US); Bradley E. PATT, Sherman Oaks, CA (US); Ian McRURY, Medway, MA (US)

(21) Appl. No.: 19/327,810

(22) Filed: Sep. 12, 2025

Related U.S. Application Data
(60) Provisional application No. 63/694,538, filed on Sep. 13, 2024.

(51) Int. Cl. A61L 27/36; A61F 2/30

(52) U.S. Cl. CPC A61L 27/3608; A61F 2/30756; A61F 2002/30224; A61F 2002/30593; A61F 2002/30757; A61F 2002/30761; A61L 2430/02

(57) ABSTRACT

A method of repairing a chondral or osteochondral defect using an implant composition made of a plurality of demineralized bone fibers formed into a contiguous shape having a peg portion and a sheet portion to facilitate cartilage repair.

### CROSS-REFERENCE TO RELATED APPLICATION(S)

This application claims priority to and the benefit of U.S. Provisional Application No. 63/694,538, filed Sep. 13, 2024, the entire content of which is herein incorporated by reference.

### BACKGROUND

The tissues that compose and support joint surfaces are collectively called osteochondral tissues. The most superficial of these, the articular cartilage, is essential for absorbing shock and maintaining normal joint environment. Regardless of the cause, the degeneration of articular cartilage can result in irreversible osteoarthritis. While this loss of cartilage is the most noticeable effect of osteoarthritis, there are also many changes to the underlying bone caused by injury and osteoarthritis. In degenerated joints there is a thickening, or sclerosis of the bone and frequently the formation of bone marrow lesions. Nutrition to these tissues is provided by the bone marrow, and disruption of the tissues may compromise nutrition causing further degeneration.

Debate continues as to the initial event(s) that causes osteoarthritis. Some investigators contend that articular cartilage changes precede subchondral bone changes; while other investigators contend the opposite. It appears that changes in articular cartilage and subchondral tissues are intertwined such that distinguishing a single initiating change in either tissue is difficult. Nonetheless, it is clear that any strategies for repair of cartilage defects or cartilage bone defects must account for changes to the bone.

While a number of medical devices have been developed to facilitate the repair of osteo and osteochondral defects, there is a need for an implant capable of stimulating repair of osteochondral injuries.

A further issue exists in the treatment of cartilage injuries in that the articular cartilage is generally less than 4 mm thick and fixation of any implant into a lesion is difficult.

### SUMMARY OF INVENTION

The inventors of the presently disclosed subject matter have discovered an advantageous implant for stimulation of repair of osteochondral or chondral defects. The presently disclosed implant is capable of: 1) providing an implant that is both osteo and chondro-inductive and osteo chondro conductive to facilitate regeneration of a chondral or osteochondral defect, 2) augmenting the effectiveness of the blood clot formed during a microfracture treatment and 3) self-stabilizing during surgery unlike other implants. Accordingly, aspects of embodiments of the present invention are directed to a means of improving the regeneration of tissue through the use of an implant, which is composed of fibers of demineralized bone and formed into an appropriate shape.

In one embodiment of the invention, the implant made of a plurality of fibers has a peg portion and a sheet portion. According to some embodiments of the present invention the peg portion of the implant is placed in a cavity formed in the bone that acts with dual purpose of both fixing the implant in place and allowing a conduit for bone marrow to be absorbed into the whole implant. The bone marrow is then retained in the defect site where it can stimulate a repair of the tissue with the bone morphogenic proteins that are present in the demineralized bone fibers providing molecular signals to stimulate and enhance the repair.

In some embodiments of the invention the implant is cannulated allowing access of bone marrow elements to the healing region of the cartilage.

In some embodiments of the present invention, the implant has a cylindrical top and a central peg and is placed like a thumb tack into an implantation site that has been prepared to be the same diameter as the implant.

In some embodiments the implant is a larger sheet with multiple pegs that is trimmed to the size and shape of the defect by the surgeon prior to implantation.

In specific embodiments, the length of the peg portion of the implant is selected from about 5 to about 25 millimeters (mm). For example, the length of the peg portion is 5, 10, 15, 20, or 25 mm. The diameter of the peg portion is selected from a range of about 2 to about 5 mm. For example, the diameter of the peg portion is 2, 2.5, 3, 3.5, 4, 4.5, or 5 mm.

The peg portion may be tapered with the greater diameter being at the proximal (sheet ) end of the implant.

In other specific embodiments, the sheet portion of the implant may be shaped to facilitate use of multiple implants to cover a larger defect area. For example, the overall sheet portion surrounding the region of the peg portion may be in a shape of or similar to a rectangular, a square, a circle, or a non-perfect shape thereof and having a perimeter that forms a rectangle, a square, a circle, or an irregular shape thereof. Additionally, the sheet portion may not form a particular polygon shape, but instead may form a perimeter of straight sides. The straight sides may each independently have a length selected from a range of about 5 and about 20 mm. For example, the straight sides each independently have a length of 5, 6, 7, 8, 9, 10, 11, 12, 13, 14, 15, 16, 17, 18, 19, or 20 mm. In example embodiments, in which the sheet portion has a circular shape, the diameter of the circular shape may be selected from a range of about 5 and 20 mm. For example, the diameter of the circular shape is 5, 6, 7, 8, 9, 10, 11, 12, 13, 14, 15, 16, 17, 18, 19, or 20 mm. In additional or alternative embodiments, the sheet portion may have a thickness selected from a range of about 0.5 to about 5 mm. For example, the sheet portion may have a thickness of 0.5, 1, 1.5, 2, 2.5, 3, 3.5, 4, 4.5, or 5 mm.

In other specific embodiments, the sheet portion tesselates, i.e. the sheet portion forms complete coverage of a surface. Exemplary shapes that tesselate are squares, triangles, and hexagons.

The implant made of a plurality of fibers as described herein having a peg portion and a sheet portion, may be made of demineralized bone fibers (DBF).

In other embodiments of the invention the implant is a sheet of fibers that is implanted in the dry, lyophilized state to encourage hydration in situ, and to allow the peg portion which is stiffer when dry to be more easily inserted into the bone cavity.

In other embodiments of the invention the implant is a sheet of fibers, wherein one face of the sheet has a smoothed and densified surface and the other face is lower density and more open to encourage infiltration of blood and bone marrow.

In other embodiments of the invention the implant is a cylinder that is selected from a range of about 3 to 15 mm in diameter and is selected from a range of about 3 to 45 mm in length; and is designed to fit into a cylindrical osteochondral implantation site, which will expand upon hydration to swell and remain in place.

In other embodiments of the invention, the cylindrical implant has a densified surface.

The inventive subject matter also includes a method of repairing an osteochondral or chondral defect using the implant as disclosed herein. The method includes preparing the defect site by trimming loose or fibrillated cartilage, performing a microfracture to create a bleeding bone bed, including using a pick with dimensions suitable to provide a cavity in the bone of the same dimensions as the peg, placing the lyophilized/dry implant into the site and allowing it to hydrate with the bone marrow released by the microfracture procedure.

The inventive subject matter also includes a method of repairing an osteochondral or chondral defect using the cylindrical implant as disclosed herein. The method includes preparing the defect site by trimming loose or fibrillated cartilage, drilling or using a tubular chisel to create a cylindrical defect, placing the lyophilized/dry cylindrical implant into the site and allowing it to hydrate with the bone marrow released by the defect site preparation.

The inventive subject matter also includes a method for making the presently disclosed implant. In typical embodiments, the method for making the presently disclosed implant includes dispersing a plurality of fibers in fluid and injecting into a vented mold thereby draining the fluid and filling the plurality of fibers in the mold.

The method may further include heating the plurality of fibers in the mold. Heating of the plurality of fibers may occur at or between 45 to 55 degrees Celsius. The heating may be preferentially applied to one surface of the implant. In example embodiments, the fibers (e.g., demineralized bone fibers) are lyophilized.

### BRIEF DESCRIPTION OF THE DRAWINGS

FIG. 1 shows an implant (1) with a round sheet top (2) and a peg region (3).

FIG. 2 shows a variant of the implant (1) wherein the top portion (2) is a square shape that can be tessellated to cover larger defects.

FIG. 3 shows a variant of the implant (1) wherein the top portion (2) is a hexagonal shape that can be tessellated to cover larger defects.

FIG. 4 shows a variant of the implant (1) wherein the top portion (2) is a rectangular shape.

FIG. 5 shows a variant of the implant (1) wherein the top portion (2) is a round shape and the implant is cannulated (4).

FIG. 6 shows a variant of the implant (1) wherein the top portion (2) is a rectangular shape and the implant is cannulated (4).

FIG. 7 shows a variant of the implant (1) wherein the top portion (2) is a hexagonal shape that can be tessellated to cover larger defects, and wherein the implant is cannulated (4).

FIG. 8 shows a variant of the implant (1) wherein the top portion (2) is a square shape that can be tessellated to cover larger defects, and wherein the implant is cannulated (4).

FIG. 9 shows an apparatus for making non cannulated implant (1) using the water assisted injection molding process. A plunger (5) is in a syringe barrel (6) wherein DBF fibers (7) are dispersed in a fluid. The syringe barrel is attached to an adapter (8) that connects to a detachable mold (9) that has vents (10) and a cavity the shape of the implant (1)

FIG. 10 shows an apparatus for making a cannulated implant (1) using the water assisted injection molding process. A plunger (5) is in a syringe barrel (6) wherein DBF fibers (7) are dispersed in a fluid. The distal end of the syringe barrel is attached to an adapter (8) that connects to a detachable mold (9) that has vents (10) and a cavity the shape of the implant (1). A rod (11) defines the cannulation.

FIG. 11 shows a variant of the implant (12) in the form of a truncated cone.

FIG. 12 shows a variant of the implant (12) in the form of a truncated cone with a cannulation (13).

FIG. 13 shows a variant of the implant (14) in the form of a cylinder.

FIG. 14 shows a variant of the implant (14) in the form of a cylinder with a cannulation (13).

FIG. 15 shows a mold (15) for making a sheet (16) of DBF from which shaped implants may be cut.

FIG. 16 shows an awl (17) suitable for preparation of an implant site. The awl has a region (18) to create the implantation site for the peg region of the implant and a cutting feature (19) to create a recess to accommodate the sheet portion of the implant.

FIG. 17 shows an instrument for implantation of a cannulated implant. A handle (20) is at the proximal end of the instrument and a shaft (21) at the distal end. The implant (1) is an interference fit on the shaft (21).

FIGS. 18A, 18B, 18C show the steps in the implant implantation. FIG. 18A shows a cartilage defect (22) in a knee femur. FIG. 18B shows creation of the implantation site using the awl (17). FIG. 18C shows the implant in place.

### DETAILED DESCRIPTION

Aspects of the embodiments of the present invention are directed to an approach for augmenting the repair and healing of osteochondral and chondral defects using demineralized bone fiber (DBF) implants.

As used herein, a chondral defect refers to a focal area of damage to the articular cartilage (the cartilage that lines the end of the bones). An “osteochondral defect” refers to a focal area of damage that involves both the cartilage and a piece of underlying bone. These can occur from an acute traumatic injury to the knee or an underlying disorder of the bone.

In some aspects, embodiments of the present invention include DBF implants, methods of forming DBF implants, and kits including suitably shaped and sized DBF implants for repair of focal cartilage defects.

As used herein “implant,” “DBF implant,” “implant of the present disclosure,” and like terms are used interchangeably to refer to a suitably shaped demineralized bone fiber implant made using demineralized bone fibers (DBF) as disclosed herein and disclosed in U.S. Pat. Nos. 9,486,557 and 9,572,912, and WO 2016/123583, the entire contents of all of which are incorporated herein by reference. For example, as shown throughout the present disclosure, suitably shaped DBF implants include a sheet, a cylinder, and a contiguous shape of a sheet having a peg or multiple pegs designed to assist fixation and to provide a conduit for the hydration of the implants with blood and bone marrow.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 5%, 4%, 3%, 2%, 1%, 0.5%, or 0.05% of a given value or range.

The inventors of the presently disclosed subject matter have discovered an advantageous implant that is capable of: 1) serving as a patch for the chondral or osteochondral defect site to be repaired, and 2) self-stabilizing during surgery unlike other implants. Accordingly, aspects of embodiments of the present invention are directed to a means of improving the fixation of implants and tissue to bone through the use of an implant, which may, for example, be composed of fibers of demineralized bone and formed into an appropriate shape. In particular, the implant made of a plurality of fibers has peg portion and sheet portion.

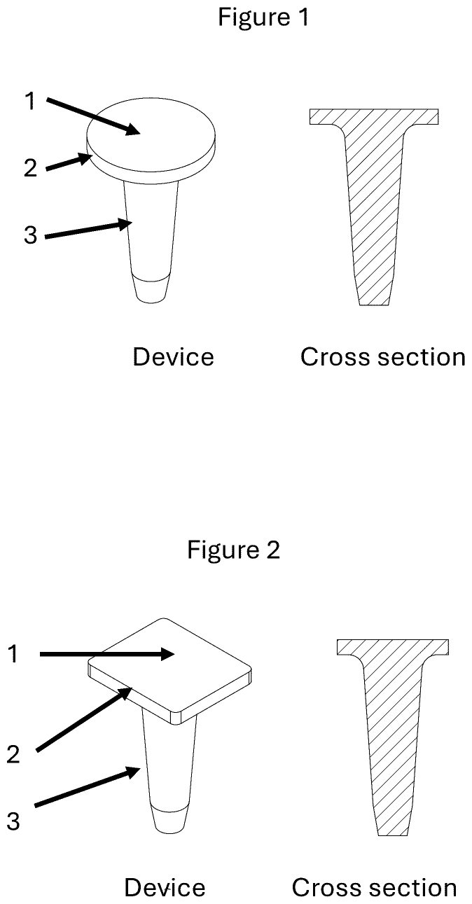

An example of an implant of the invention is shown in FIG. 1. The sheet region (2) of the implant is held in place after implantation by the peg portion (3). To facilitate implantation using arthroscopic surgery the diameter of the sheet portion is generally less than 12 mm and preferably less than 10 mm in diameter.

In specific embodiments, the length of the peg portion of the implant is selected from a range of about 5 to 25 millimeters (mm). For example, the length of the peg portion is 5, 10, 15, 20, or 25 mm. The diameter of the peg portion is selected from a range of about 2 to 5 mm. For example, the diameter of the peg portion is 2, 2.5, 3, 3.5, 4, 4.5, or 5 mm.

The peg portion may be tapered with the greater diameter being at the proximal (sheet) end of the implant.

In example embodiments, in which the sheet portion has a circular shape, the diameter of the circular shape may be selected from a range of about 5 to 20 mm. For example, the diameter of the circular shape is 5, 6, 7, 8, 9, 10, 11, 12, 13, 14, 15, 16, 17, 18, 19, or 20 mm. In additional or alternative embodiments, the sheet portion may have a thickness is selected from a range of about 0.5 to 5 mm. For example, the sheet portion may have a thickness of 0.5, 1, 1.5, 2, 2.5, 3, 3.5, 4, 4.5, or 5 mm.

In example embodiments, the overall sheet portion surrounding the region of the peg portion may be in a shape of or similar to a rectangular, a square, a circle, or a non-perfect shape thereof and having a perimeter that forms a rectangle, a square, a circle, or an irregular shape thereof. Additionally, the sheet portion may not form a particular polygon shape, but instead may form a perimeter of straight sides. The straight sides may each independently have a length selected from a range of about 5 to 20 mm. For example, the straight sides each independently have a length of 5, 6, 7, 8, 9, 10, 11, 12, 13, 14, 15, 16, 17, 18, 19, or 20 mm.

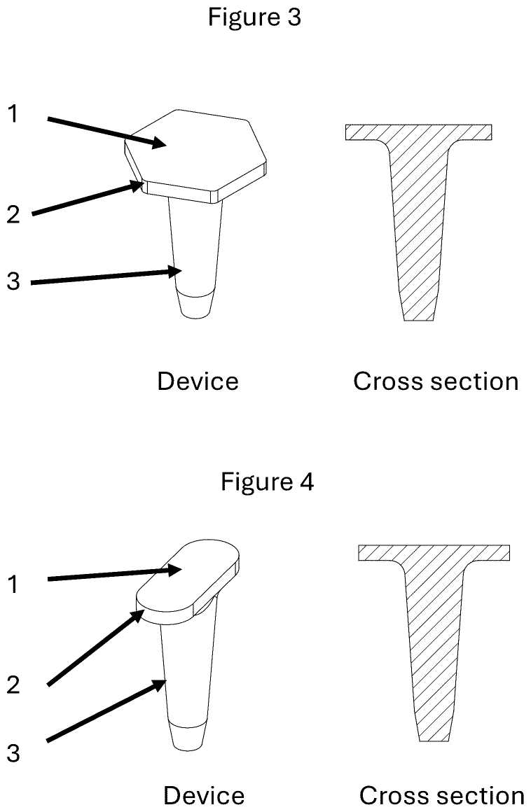

In order to treat larger defects in the cartilage multiple implants can be used. Coverage of larger defects is further enhanced by use of designs such as is shown in FIGS. 2 and 3 where the top portion (2) is a shape that can be tessellated to provide total coverage of the defect. Non limiting examples of shapes that can be tesselated are a square (FIG. 2) and a hexagon (FIG. 3).

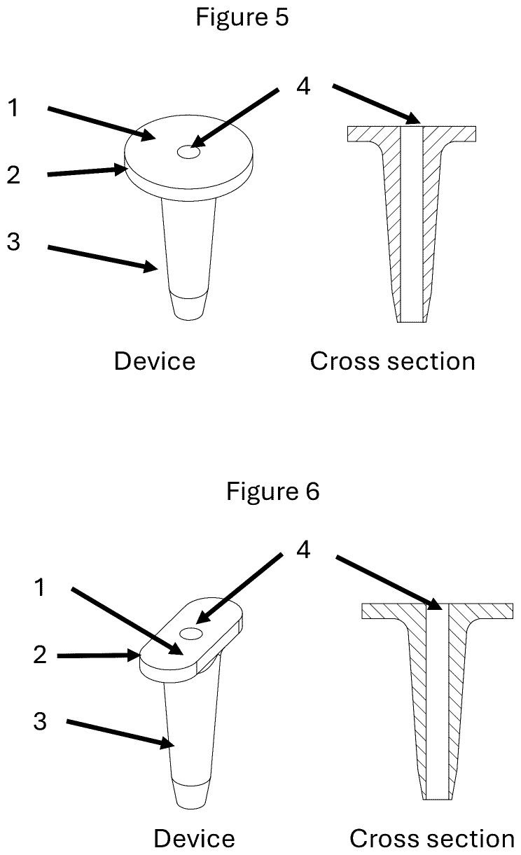

In a further embodiment of the invention the implant is cannulated as is shown in the examples in FIGS. 5 to 8. The cannulation has the benefit of providing communication with the subchondral bone marrow allowing beneficial elements such as cells to travel up the implant to the cartilage region, and to facilitate remodeling of the demineralized bone fiber of the implant through endochondral ossification into new bone in the peg region of the implant and into cartilage in the cartilage region.

The cannulation of the implant may be selected from about 0.5 to 3 mm in diameter.

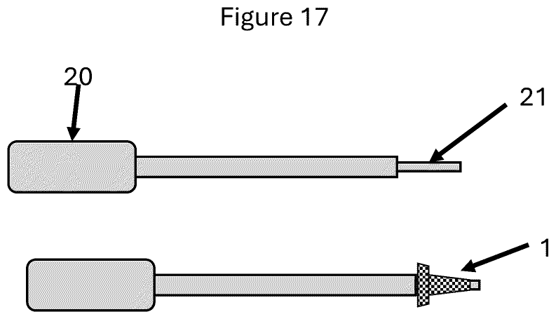

A further benefit of the cannulated implant is that a delivery instrument such as is shown in FIG. 17 can be used to deliver the implant into the surgically created implantation site.

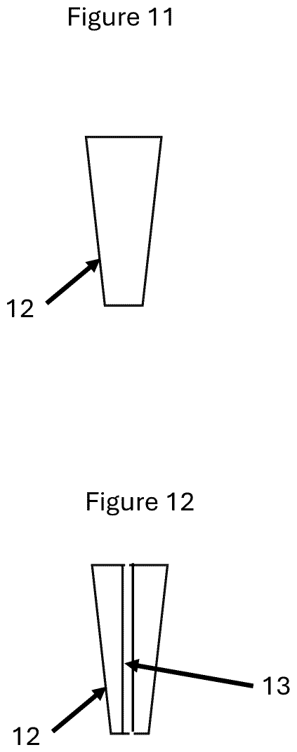

In a further embodiment of the invention the implant may be in the form of a truncated cone (12) as shown in FIG. 11, or of a truncated cone (12) with a cannulation (13) as is shown in FIG. 12. The larger diameter of the cone is designed to be at the proximal end of the implant with the taper intended to make insertion into a surgically created defect easier. The top diameter of the cone is in the range of about 4 mm to about 15 mm and the reduction in diameter at the distal end is about 0.5 to about 3 mm. The length of the implant is from about 5 mm to about 20 mm. For the cannulated version of the implant the cannulation of the implant may be from about 0.5 to about 3 mm in diameter.

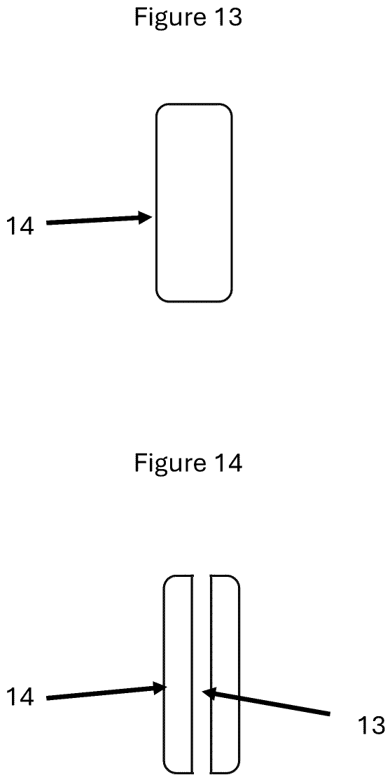

In a further embodiment of the invention the implant may be in the form of a cylinder (14) as shown in FIG. 13, or of a cylinder (14) with a cannulation (13) as is shown in FIG. 14. The diameter of the cylinder is in the range of about 4 mm to about 15 mm. The length of the implant is from about 5 mm to about 20 mm. For the cannulated version of the implant the cannulation of the implant may be from about 0.5 to about 3 mm in diameter. The distal tip of the cylinder may be rounded.

The popularity of demineralized bone matrix (DBM)-based products is based on the ability to induce bone formation through expression of inherent non-collagenous proteins that stimulate some cell types present at the graft site to differentiate into bone forming cells. This induction of bone formation process is referred to as “osteoinduction” and is due to the natural presence of bone morphogenic proteins (BMPs). DBM also provides a scaffold for these cells to populate and spread throughout in a process known as “osteoconduction.” Demineralized bone in the form of a fiber, known as Demineralized Bone Fiber (DBF) has a physical form that has been shown to optimize and enhance the osteoconductive performance of DBM. In some embodiments of the present invention, a composition and method of manufacture of DBF fibers is as disclosed in U.S. Pat. Nos. 9,486,557 and 9,572,912, supra. When DBM or DBF is combined with osteogenic cells that are capable of forming bone, the three mechanisms of bone healing (e.g., osteoinduction, osteoconduction, and osteogenesis) are combined.

While it is well known that these materials can be used to stimulate bone formation they have not been used for repair of articular cartilage defects, in part due to the lack of a suitable form of implant and in part due to a belief that the bone morphogenic proteins present in the DBF will generate bone rather than cartilage. It is a particular aspect of the Demineralized Bone Fibers DBF used herein that they are sufficiently long that they can be formed into entangled shapes that may also be treated such that their cohesiveness is enhanced. Furthermore, the geometry of the implant provides a peg that can facilitate holding the disc of material in the cartilage region of the defect in place, and that in its dry state that the implant is sufficiently rigid for it to be pressed into place in the same manner as a thumb tack. A further benefit of implantation of the implant in the dry state is that it will be hydrated by the fluid present in the implantation site imbibing the implant with beneficial agents present in the blood and bone marrow.

Without being bound to any theory, it is hypothesized that bone can be formed by endochondral ossification, i.e. through formation of cartilage prior to bone formation and that endogenous signals in the injury site provide signals to the tissue to remain in the cartilage stage.

The DBF implant is dried so that it has sufficient rigidity to allow it to be pushed into a pre-formed hole. The DBF fibers may be easily formed into any of the required implant shapes using molding or wet laying processes prior to drying. Optionally a heating step may be utilized which has been shown to impart even greater cohesion to formed DBF implants without affecting the implant's osteoinductivity. Optionally, the heating step may be preferentially applied to the surface of the device to provide densification, smoothing, and greater cohesion.

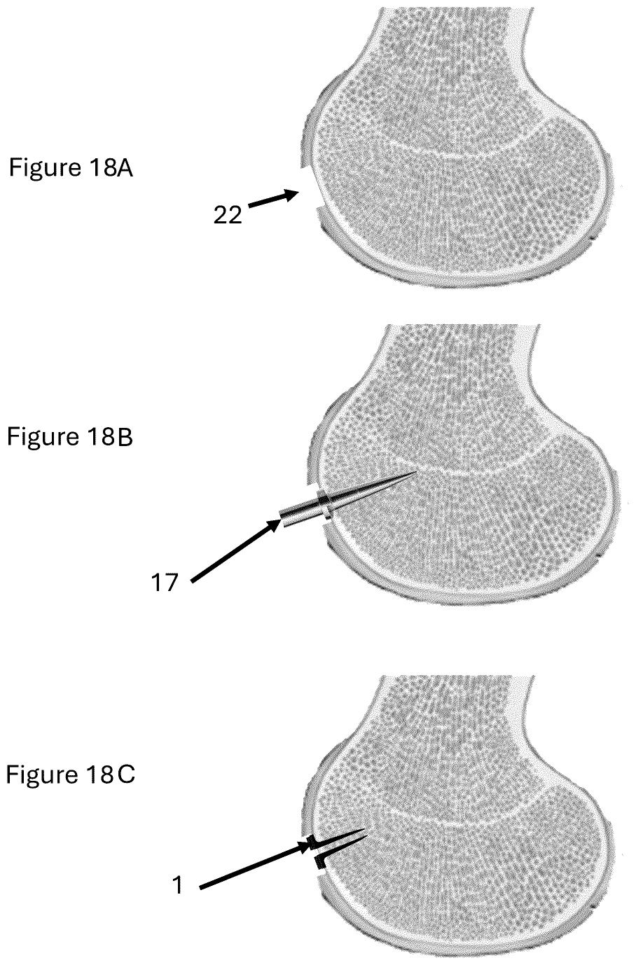

The steps of the surgical technique are shown in FIG. 18. It is expected that the procedure is performed arthroscopically. The first step is to remove any loose or damaged cartilage. From an assessment of the size of the defect the surgeon may decide what size and shape, or if multiple implants are required. For the implant design of FIGS. 1 to 8 an awl (17) is used to produce a tapered hole to receive the implant. The surgeon may use the cutting feature (19) to define how far into the subchondral bone the implant should sit. It is preferred that the top of the implant sits either flush with the articular surface or slightly below. For a cannulated implant the Introducer shown in FIG. 17 may be used.

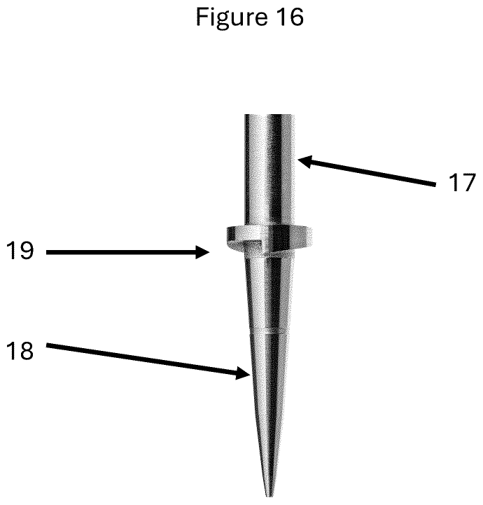

The hole to receive the peg portion of the implant may be formed by drilling, tapping, or by use of an awl. An example of a suitable awl is provided in FIG. 16 where the distal portion of the awl (18) is dimensioned to match the peg portion of the implant, and an optional cutting feature (19) can be used to cut a recess in the bone to accommodate the sheet portion of the implant.

Implants shaped as a truncated cone may be inserted into holes created using an awl or tapered drill.

Cylindrical implants may be inserted through a cannula, or tube and placed into a surgically created cavity using a push rod or plunger.

The implant may be supplied as a kit with an awl/pick to form the cavity for the peg portion of the implant. The awl/pick may be supplied as sterile disposable single use item or may be designed to be re-usable.

The implant is dependent on having some rigidity to enable it to be inserted in the bone and as such this is best achieved by using the DBF in a dry state. Once in place in the bone the fibers will hydrate. The effect of this will be to cause them to swell, providing additional fixation.

In some embodiments of the present invention, the DBF used in an implant uses bone that has had the mineral component removed by a demineralization process that renders the graft malleable and not hard. The bone is then further formed into fibers by cutting along the long axis such that the collagen fibers within it are maintained in their natural fibrous form, as disclosed in U.S. Pat. Nos. 9,486,557 and 9,572,912, supra.

A number of methods of forming cylindrical implants from DBF are also disclosed in WO 2016/123583, the entire content of which is herein incorporated by reference.

In some embodiments, the methods for making the bone fibers include demineralizing whole bone and subsequently cutting the demineralized bone in a direction parallel to the orientation of collagen fibers within the demineralized bone to form elongated bone fibers. The bone material of the present invention is derived from human (allograft) or animal (xenograft) cortical bone and is processed in such a manner to provide grafts of high utility based on the controlled geometry of the bone fibers. For veterinary applications bone from the same species. e.g., canine for canine patients (allograft) may be used as well as bone from other species (xenograft). It will be obvious to one skilled in the art that fibers other than demineralized bone fibers may be utilized to make a bone graft of this invention. Such fibers may be made from resorbable polymers or bioactive glasses or mixtures thereof, and may be used in place of or as an additive to the demineralized bone fibers (DBF). The methods of preparation of the graft provide improved efficiency and uniformity with reproducible results and decreased requirements for equipment and resulting costs. The implant device forms according to some embodiments of the present invention do not require the addition of exogenous materials to maintain the form of the graft. These improved characteristics will be apparent to one skilled in the art based upon the present disclosure.

It is also important, in various applications, that the DBF can be dried to render a stiffer implant at the time of implantation.

A further benefit of the DBF fibers is their ability to be processed to form an implant that retains its integrity when wet. This is especially important as in order to be suitable for use in arthroscopic surgery implants must retain their shape and integrity when placed into an aqueous emvironment.

### Processing of Fibers

Processing of the demineralized bone fibers to produce a desired shape or form of the bone fibers may be performed using any suitable method. To make some of these forms, the bone fibers may be collected, ideally in their hydrated state, and compressed using pressure molds, the pressure being sufficient to form the required shape but not so high as to lose the porosity of the fibrous structure. In some embodiments, the bone fibers are formed using a wet lay technique as is well understood by those skilled in the art of nonwoven or paper manufacture. Using a wet lay technique, the cut bone fibers are suspended in an aqueous solution to form a bone fiber slurry. Any suitable biocompatible aqueous solution may be used. Non-limiting examples of biocompatible aqueous solutions include: water, saline, and/or solutions including salts such as phosphate buffered saline (PBS), Ringer's solution, Lactated Ringer's solution, and saline with 5% dextrose. In some embodiments of the present invention, cut fibers are placed into saline to create a slurry of entangled bone fibers. The bone fiber slurry is suspended over a mesh screen (having holes) and the saline is drained resulting in a wet lay process, such that a sheet of demineralized bone fibers is formed on the mesh screen. The screen may be contoured to provide a three dimensional shape to the screen such as indentations that for the peg features may be directly produced, or is flat so that a sheet is produced. The resulting devices may be then dried using heat and/or vacuum or other means such as lyophilization (freeze-drying). In some embodiments, prior to drying, the sheet is placed in a mold and compressed to a defined thickness and shape, followed by drying. As discussed herein, density, porosity and overall dimensions of the resulting product may be controlled using various molds and techniques.

Hydrated fibers may also be simply placed into a cylindrical mold cavity and lightly compressed using a plunger or push rod. A set amount of fiber is introduced into a cylindrical mold and the plunger used to compress the fibers to the required density through control of the depth that the plunger is pushed.

In some embodiments a vacuum oven is used, whereby the application of vacuum removes moisture and dries the implant. Alternatively or additionally, lyophilization is used.

In some embodiments the heating step is undertaken by placing the implant in contact with a metal or other high heat-conductivity surface such that the degree of annealing/crosslinking is enhanced at that surface.

In other embodiments, the bone fibers are further processed in a second drying step that may include vacuum drying and/or lyophilization.

In other embodiments the bone fibers may retain some moisture and will be placed in moisture impervious packaging.

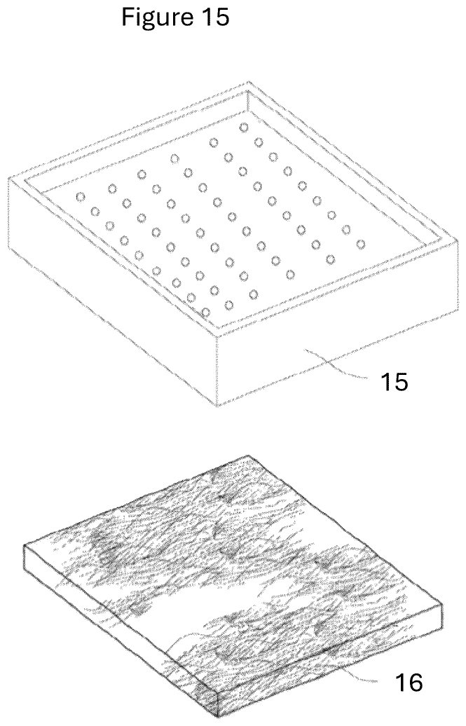

A simple mold of the sort shown in FIG. 15 may be used to make DBF sheets of 0.5 mm to 5 mm thick, where the mold lid may be placed on the mold (15) (the mold having holes for drainage of the liquid in the DBF slurry) where the lid is in contact with the DBF after the DBF has been wet laid and may define the degree of compression of the DBF and hence the density of the sheet.

The mold may also have depressions in its base to allow pegs to be created.

A DBF sheet that is dried will have a low wet strength when rehydrated and improvement to the DBF sheet wet strength may be affected by placing the mold in an oven at 45 to 55° C. and heat treating the sheet for up to 2 hours.

In one embodiment the mold lid is made from a material with higher heat conductivity than the base of the mold. For example, the mold base is polyether ether ketone (PEEK) while the lid is stainless steel. The lid may be pre heated to 45-55C and placed onto the mold in order that one surface of the sheet is preferentially modified. The mold with lid is optionally placed into an oven at a temperature of between about 45° to 55° C.

While wet lay techniques may be used for the manufacture of different shapes from the bone fibers, it will be recognized that any other molding or forming technique used with textile fibers could be used. Fibers with and without excipients may be directly molded using compression into any shape. In some embodiments excipients are selected that enhance the lubricity of the implant facilitating delivery and further reducing friction or binding during this procedure.

The wet lay process was originally developed for use in paper making and textiles where the fibers are processed to make a two-dimensional sheet-like product. As the fluid drains the fibers are laid onto the surface of the mold and as such are in a plane that is generally parallel to the plane of the sheet being produced. While this process can accommodate some undulations and be used to make shapes like egg cartons it is wholly unsuitable for the fabrication of cylindrical shapes.

According to embodiments of the present invention, by dispersing fibers in an excess of fluid, the fluid and fibers may be directed into molds of small diameter and long length. Implants that have a diameter selected from about 2 to 5 mm and a length of about 5 cm have a volume of 0.15 cm³ to 0.98 cm³. The required mass of DBF to fill those molds is approximately 0.15 gram to 1 gram; and, in one example, the mass of DBF is dispersed in about 20 mls of fluid in a syringe. Any suitable fluid buffer may be used. For example, the DBF fibers may be dispersed in phosphate buffered saline (PBS), water, or any biocompatible buffer or liquid.

In contrast to methods of fabrication of the prior art, rather than rely on gravitational flow, an elevated pressure is applied to the dilute fiber and fluid dispersion. This forces the fibers to flow down narrow diameter structures rather than form an entangled clump at the entrance to the mold cavity.

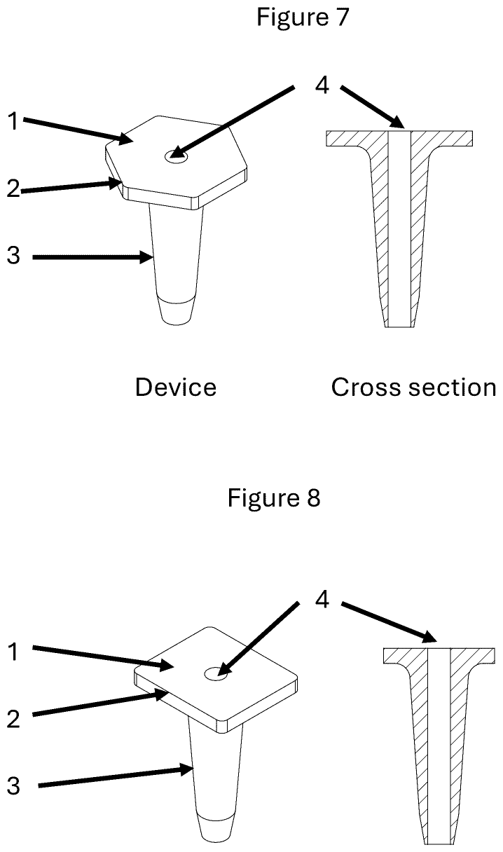

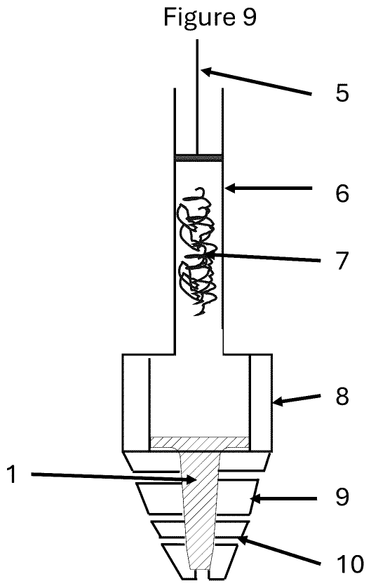

FIG. 9 depicts an apparatus for water-assisted injection molding of DBF fibers. The required mass of DBF fibers (7) are loaded into a syringe (6). A suitable fluid (e.g., PBS) is then added to the syringe. The distal end of the syringe is then fitted into an adapter (8) to which is attached a detachable mold (9). The mold is the required dimensions of the implant (1) to be made. As with conventional injection molding, the cylinder (FIG. 7) has a taper or draft to allow removal of the molded part. The mold is tapered towards its distal end and has vents (10) along its length. By suspending the DBF in a fluid application of downward pressure on the plunger (5) causes the slurry to be transported down the syringe barrel into the mold. The fluid exits the mold through the vents (10) leaving the DBF in the mold. The detachable mold is removed after DBF injection and placed into an oven or lyophilizer for drying. Optionally a cap (not shown) can be placed on the top of the mold. Multiple molds may be used with one adapter and syringe to allow multiple parts to be fabricated.

In some embodiments of the present invention, the ratio of fluid to DBF is selected from a range of about 5 mls to about 1 gram. In other embodiments, the ratio of fluid to DBF is about 10 mls to 1 gram. In still other embodiments, the ratio of fluid to DBF is greater than about 200 mls fluid to 1 gram DBF.

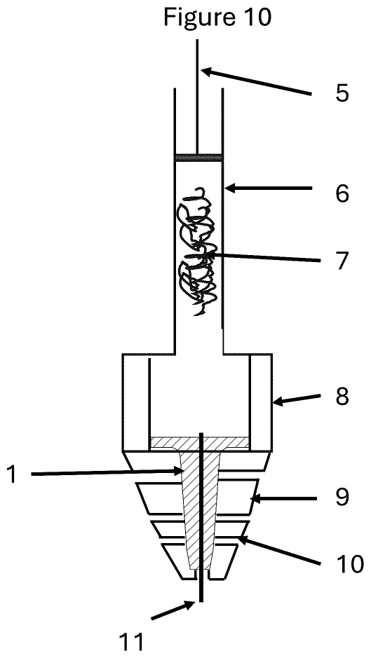

The water assisted injection molding process may also be used to make cannulated implants such as the device 1 shown in FIG. 10. The design and operation of the system are the same as the system shown in FIG. 9, differing in the placement of a rod (11) in the mold prior to injecting the DBF. The rod forms the cannulation and is removed after drying.

### Excipients and Additives

Additives are contemplated to modify biological or other properties of the implant according to embodiments of the present invention. Non-limiting examples of additives include growth factors such as bone morphogenetic proteins (BMPs), including BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, and BMP-18; Vascular Endothelial Growth Factors (VEGFs), including VEGF-A, VEGF-B, VEGF-C, VEGF-D and VEGF-E; Connective Tissue Growth Factors (CTGFs), including CTGF-1, CTGF-2, and CTGF-3; Osteoprotegerin, Transforming Growth Factor betas (TGF-βαs), including TGF-β-1, TGF-β-2, and TGF-β-3, and inhibitors for tumor necrosis factor (e.g., anti-TNF-α). Morphogens may also include Platelet Derived Growth Factors (PDGFs), including PDGF-A, PDGF-B, PDGF-C, PDGF-D, and GDF-5; rhGDF-5; and LIM mineralization protein, insulin-related growth factor-I (IGF-I), insulin-related growth factor-II (IGF-II), fibroblast growth factor (FGF) and beta-2-microglobulin (BDGF II), as disclosed in the U.S. Pat. No. 6,630,153, the entire contents of which is incorporated herein by reference. The polynucleotides encoding the same may also be administered as gene therapy agents. The preferred bioactive substances are the recombinant human bone morphogenetic proteins (rhBMPs) because they are available in relatively unlimited supply and do not transmit infectious diseases. In some embodiments, the bone morphogenetic protein is a rhBMP-2, rhBMP-4, rhBMP-7, or heterodimers thereof. BMPs are available from Wyeth, Madison, N.J., and may also be prepared by one skilled in the art as described in U.S. Pat. No. 5,366,875 to Wozney et al.; U.S. Pat. No. 4,877,864 to Wang et al.; U.S. Pat. No. 5,108,922 to Wang et al.; U.S. Pat. No. 5,116,738 to Wang et al.; U.S. Pat. No. 5,013,649 to Wang et al.; U.S. Pat. No. 5,106,748 to Wozney et al.; and PCT Patent Nos. WO93/00432 to Wozney et al.; WO94/26893 to Celeste et al.; and WO94/26892 to Celeste et al., the entire contents of all of which are herein incorporated by reference.

In some embodiments of the present invention, the implant is longer than the depth of the hole to be treated and in these instances the surgeon may cut the implant to a desired length.

In some embodiments of the present invention, implants are formed and stored in tubes.

### EXAMPLES

The following examples use cortical human bone. As discussed herein, either human or animal bone may be used as a source of cortical bone. Fibers were produced using the methodology described in U.S. Pat. Nos. 9,486,557 and 9,572,912, supra.

### Example 1

A number of molds were produced to allow fabrication of an implant of the design shown in FIG. 5 using the Water Assisted Injection Molding (WAIM) process. The molds were designed to be two pieces that clipped together. The peg portion of the implant cavity was 12 mm long with a diameter tapering from 4 mm to 3 mm along its length with a top portion 9 mm in diameter and 2 mm thick. A cannulating rod with a diameter of 1.5 mm was held in place by the distal part of the mold. 0.5 grams of DBF was suspended in 25 ml of distilled water in a syringe and injected into the mold. The mold was removed from the syringe and a cap placed on it. The process was repeated multiple times to produce a number of implants. The molds were placed in an oven at 55° C. for 90 minutes and then into a vacuum oven at 20° C. and were dried overnight. After removal from the oven the cannulating rods were removed and the implants were removed from the molds.

### Example 2

A number of molds were produced to allow fabrication of an implant of the design shown in FIG. 6 using the Water Assisted Injection Molding (WAIM) process. The molds were designed to be two pieces that clipped together. The peg portion of the implant cavity was 12 mm long with a diameter tapering from 4 mm to 3 mm along its length with a top portion having a 9 mm diameter and a thickness of 2 mm. A cannulating rod with a diameter of 1.5 mm was held in place by the distal part of the mold. 0.5 grams of DBF was suspended in 25 ml of distilled water in a syringe and injected into the mold. The mold was removed from the syringe and a cap placed on it. The process was repeated multiple times to produce a number of implants. The molds were placed in an oven at 55° C. for 90 minutes and then into a vacuum oven at 20° C. and were dried overnight. After removal from the oven the cannulating rods were removed, and the implants were removed from the molds.

### Example 3

An implant from those produced in Example 2 was placed into a beaker containing sterile water. Examination of the implant after 17 hours showed that it retained its shape and integrity, making it suitable for use in arthroscopic surgery.

### Example 4

45 grams of DBF fiber were wet laid in a 10 cm×11 cm flat mold to produce a sheet of DBF approximately 3 mm thick. The mold was heated at 55° C. for two hours to bond the fibers and dry the sheet. The sheet was cut into 10 mm diameter discs suitable for use as implants to treat chondral and osteochondral defects.

### Example 5

45 grams of DBF fiber were wet laid in a 10 cm×11 cm flat mold to produce a sheet of DBF approximately 3 mm thick. A stainless-steel mold lid, pre-heated to 55° C. was placed on the mold. The mold was placed in an oven set at 55° C. for two hours to bond the fibers and dry the sheet. The sheet was cut into 10 mm diameter discs suitable for use as implants to treat chondral and osteochondral defects.

### Example 6

A number of molds were produced to allow fabrication of a cylindrical implant of the design shown in FIG. 3, as well as the designs shown in FIGS. 1, 2, 4, 5, 6, 7, and 8, using the Water Assisted Injection Molding (WAIM) process. The molds were designed to be two pieces that clipped together. The implant cavity was 6 mm in diameter and 10 mm in length. 0.35 grams of DBF was suspended in 25 ml of distilled water in a syringe and injected into the mold. The mold was removed from the syringe and a cap placed on it. The process was repeated multiple times to produce a number of implants. The molds were placed in an oven at 55° C. for 90 minutes and then into a vacuum oven at 20° C. and were dried overnight. After removal from the oven the implants were removed from the molds.

While the present invention has been illustrated and described with reference to certain exemplary embodiments, those of ordinary skill in the art will understand that various modifications and changes may be made to the described embodiments without departing from the spirit and scope of the present invention, as defined in the following claims.

Additionally, although relative terms such as “outer,” “inner,” “upper,” “lower,” “below,” “above,” “vertical, “horizontal” and similar terms have been used herein to describe a spatial relationship of one element to another, it is understood that these terms are intended to encompass different orientations of the various elements and components of the device in addition to the orientation depicted in the figures.

What is claimed is:

1. A method of repairing a chondral or osteochondral defect using a demineralized bone fiber (DBF) implant composition having a contiguous shape comprising a peg portion and a sheet portion, the method comprising:
creating a cavity in a bone area of a cartilage defect; and
placing the peg portion into the bone cavity, thereby placing the connected sheet portion in contact with the chondral defect.

2. The method of claim 1, wherein the DBF implant comprises a plurality of fibers cut from demineralized bone, the plurality of fibers forming the contiguous peg and sheet portions.

3. The method of claim 1, wherein the length of the peg portion is in a range from about 10 to 50 millimeters (mm).

4. The method of claim 1, wherein the diameter of the peg portion is in a range from about 3 to 10 mm.

5. The method of claim 1, wherein the sheet portion has a rectangular, square, circular, or irregular perimeter.

6. The method of claim 1, wherein the sheet portion has a perimeter of straight sides.

7. The method of claim 6, wherein each of the straight sides independently has a length in a range from about 5 to 20 mm.

8. The method of claim 1, wherein the sheet portion has a diameter in a range from about 5 to 20 mm.

9. The method of claim 1, wherein the sheet portion has a thickness in a range from about 0.5 to 5 mm.

10. The method of claim 1, wherein the implant is cannulated.

11. An implant composition for repairing a chondral defect proximal to a bone or an osteochondral defect in a bone, the composition comprising:
a plurality of fibers cut from demineralized bone, the plurality of fibers forming a contiguous shape comprising a peg portion and a sheet portion.

12. The implant composition of claim 11, wherein the length of the peg portion is in range from about 10 to 50 millimeters (mm).

13. The implant composition of claim 11, wherein the diameter of the peg portion is in a range from about 3 to 10 mm.

14. The implant composition of claim 11, wherein the sheet portion has a rectangular, square, circular, or irregular perimeter.

15. The implant composition of claim 11, wherein the sheet portion has a perimeter of straight sides.

16. The implant composition of claim 15, wherein each of the straight sides independently has a length in a range from about 5 to 20 mm.

17. The implant composition of claim 11, wherein the sheet portion has a diameter in a range from about 5 to 20 mm.

18. The implant composition of claim 11, wherein the sheet portion has a thickness in range from about 0.5 to 5 mm.

19. The implant composition of claim 11 wherein the implant is cannulated.

20. A method of cartilage repair using a demineralized bone fiber (DBF) implant composition having a cylinder shape, the method comprising:
optionally preparing a bleeding bone bed on the base of the chondral defect using a microfracture technique;
creating a cavity: i) in a bone area facing a chondral defect; or ii) in an osteochondral defect area of a bone; and
placing the implant composition into the bone cavity thereby placing the connected sheet portion in contact with the chondral defect or the osteochondral defect.

21. A kit for chondral or osteochondral repair, the kit comprising:
the implant of claim 11.

22. A kit for chondral or osteochondral repair for use with the method as set forth in claim 1, the kit comprising:
a demineralized bone fiber (DBF) implant composition having a shape selected from: i) a contiguous peg portion and sheet portion; or ii) a cylinder.
